(19) United States
(12) Patent Application Publication
(10) Pub. No.: US 2026/0130834 A1
(43) Pub. Date: May 14, 2026

(54) COSMETIC KIT COMPRISING A MIXED COSMETIC COMPOSITION WHOSE PROPERTIES CHANGE FROM A LIQUID SKIN FORM TO A CREAM FORM

(71) Applicant: AMOREPACIFIC CORPORATION, Seoul (KR)

(72) Inventor: Dongwan HA, Yongin-si (KR)

(73) Assignee: AMOREPACIFIC CORPORATION, Seoul (KR)

(21) Appl. No.: 19/365,582

(22) Filed: Oct. 22, 2025

(30) Foreign Application Priority Data
Nov. 11, 2024 (KR) 10-2024-0159015

(51) Int. Cl. A61K 8/60; A61K 8/06; A61K 8/20; A61K 8/23; A61K 8/365; A61Q 19/00

(52) U.S. Cl. CPC A61K 8/60; A61K 8/068; A61K 8/20; A61K 8/23; A61K 8/365; A61Q 19/00

(57) ABSTRACT

The present disclosure relates to a cosmetic kit comprising a mixed cosmetic composition whose properties change from a liquid toner form to a cream form. Specifically, the cosmetic kit according to the present disclosure may stably form and maintain a cream formulation even in an environment where a low temperature or a small force is applied, by properties changing from a liquid toner form to a cream form in a short time without applying a large physical force.

### CROSS REFERENCE TO RELATED APPLICATION

The present application claims priority to Korean Patent Application No. 10-2024-0159015, filed Nov. 11, 2024, the entire contents of which are hereby incorporated by this reference.

### BACKGROUND OF THE INVENTION

### Field of the Invention

The present disclosure relates to a cosmetic kit comprising a mixed cosmetic composition whose properties change from a liquid toner form to a cream form.

### Description of the Related Art

Generally, a cosmetic composition of a liquid form has a disadvantage in that when applied to a face or skin, it immediately flows down and is inconvenient to use, and a cosmetic composition of a solid form is used as a balm type form, so in order to apply to a face or skin, it needs to be scooped by hand and used. As such, when a cosmetic composition of a solid form is scooped by hand and applied, a hygienic problem may occur and stability of a formulation may be affected.

In order to overcome such limitations in terms of properties of such a formulation, in a conventional technology, in case of a cosmetic composition that maintains a solid form at a low temperature and after a predetermined time at a temperature of human skin changes its phase to a liquid form, there is a disadvantage that it changes to a liquid form at a temperature of human skin and still flows down when applied to a face or skin, and in that it must to be refrigerated and stored before application, there is inconvenience in storage. In addition, in case of an existing cosmetic composition that when rubbed after applied to skin changes its phase to a solid form, only when a force of a certain level or more is applied and rubbed several times, it changes its phase to a solid form, and compared to a general cream form, there was a limitation in that it is formed into a somewhat thin form.

### SUMMARY OF THE INVENTION

The present disclosure aims to provide a cosmetic kit capable of stably forming and maintaining a cream formulation even in an environment where a low temperature or a small force is applied, by properties changing from a liquid toner form to a cream form in a short time without applying a large physical force.

In order to achieve the above object, one embodiment of the present invention provides a cosmetic kit comprising: a first composition of a nanoemulsion form of a liquid phase including wax and oil; and a second composition of a liquid phase including one or more of a salt or a pH reducing agent, and water, in which the first composition and the second composition are separated from each other.

The cosmetic kit according to the present disclosure may stably form and maintain a cream formulation even in an environment where a low temperature or a small force is applied, by properties changing from a liquid toner form to a cream form in a short time without applying a large physical force.

### BRIEF DESCRIPTION OF THE DRAWINGS

FIG. 1 is a photograph of a first composition (left) according to one embodiment of the present invention and a mixed composition (right) obtained by dropping two drops of a second composition into the first composition.

FIG. 2 is a photograph of a state in which the first composition (left) according to one embodiment of the present invention and the mixed composition (right) obtained by dropping two drops of the second composition into the first composition, are each rubbed five times.

FIGS. 3A to 3C are photographs of a state immediately after dropping the first composition according to one embodiment of the present invention on a back of a hand (FIG. 3A), a state rubbed five times (FIG. 3B), and a state rubbed twenty times (FIG. 3C).

FIGS. 4A to 4D are photographs of a state immediately after dropping the first composition according to one embodiment of the present invention on a back of a hand (FIG. 4A), a state of a mixed composition immediately after dropping two drops of the second composition into the first composition (FIG. 4B), a state of the mixed composition rubbed five times (FIG. 4C), and a state of the mixed composition rubbed ten times (FIG. 4D).

### DETAILED DESCRIPTION OF THE INVENTION

Hereinafter, the disclosure will be described in detail.

The present invention, in one aspect, may relate to a cosmetic kit comprising a first composition of a nanoemulsion form of a liquid phase and a second composition of a liquid phase.

In one embodiment, the first composition and the second composition may be separated from each other.

In one embodiment, the first composition and the second composition may be mixed immediately before use.

In one embodiment, when the first composition and the second composition are mixed, a mixed composition may change properties from a liquid toner form to a cream form.

In one embodiment, the first composition of a liquid phase may have a viscosity of 100 to 1000 cps. Specifically, the first composition of a liquid phase may have a viscosity of 100 cps or more, 110 cps or more, 120 cps or more, 130 cps or more, 140 cps or more, 150 cps or more, 160 cps or more, 170 cps or more, 180 cps or more, 190 cps or more, 200 cps or more, 210 cps or more, 220 cps or more, 230 cps or more, 240 cps or more, 260 cps or more, 280 cps or more, 300 cps or more, 320 cps or more, 340 cps or more, 360 cps or more, 380 cps or more, 400 cps or more, 420 cps or more, 440 cps or more, 460 cps or more, 480 cps or more, 500 cps or more, 550 cps or more, 600 cps or more, 650 cps or more, 700 cps or more, 750 cps or more, 800 cps or more, 850 cps or more, 900 cps or more, or 950 cps or more, and also, the first composition of a liquid phase may have a viscosity of 1000 cps or less, 990 cps or less, 980 cps or less, 970 cps or less, 960 cps or less, 950 cps or less, 940 cps or less, 930 cps or less, 920 cps or less, 910 cps or less, 900 cps or less, 890 cps or less, 880 cps or less, 870 cps or less, 860 cps or less, 850 cps or less, 840 cps or less, 830 cps or less, 820 cps or less, 810 cps or less, 800 cps or less, 780 cps or less, 760 cps or less, 740 cps or less, 720 cps or less, 700 cps or less, 680 cps or less, 660 cps or less, 640 cps or less, 620 cps or less, 600 cps or less, 580 cps or less, 560 cps or less, 540 cps or less, 520 cps or less, 500 cps or less, 450 cps or less, 400 cps or less, 350 cps or less, 300 cps or less, 250 cps or less, 200 cps or less, or 150 cps or less.

In one embodiment, a hardness of the mixed composition of a cream form may be 10 to 70 gf/cm². Specifically, a hardness of the mixed composition of a cream form may be 10 gf/cm² or more, 12 gf/cm² or more, 14 gf/cm² or more, 16 gf/cm² or more, 18 gf/cm² or more, 20 gf/cm² or more, 21 gf/cm² or more, 22 gf/cm² or more, 23 gf/cm² or more, 24 gf/cm² or more, 25 gf/cm² or more, 26 gf/cm² or more, 27 gf/cm² or more, 28 gf/cm² or more, 29 gf/cm² or more, 30 gf/cm² or more, 35 gf/cm² or more, 40 gf/cm² or more, 45 gf/cm² or more, 50 gf/cm² or more, 55 gf/cm² or more, 60 gf/cm² or more, or 65 gf/cm² or more, and a hardness of the mixed composition of the cream form may also be 70 gf/cm² or less, 68 gf/cm² or less, 66 gf/cm² or less, 65 gf/cm² or less, 64 gf/cm² or less, 62 gf/cm² or less, 60 gf/cm² or less, 58 gf/cm² or less, 56 gf/cm² or less, 54 gf/cm² or less, 52 gf/cm² or less, 50 gf/cm² or less, 45 gf/cm² or less, 40 gf/cm² or less, 35 gf/cm² or less, 30 gf/cm² or less, or 28 gf/cm² or less.

In one embodiment, the first composition and the second composition may be mixed in a weight ratio of 15-25:1. Specifically, the weight ratio of the first composition to the second composition may be 15:1 or more, 16:1 or more, 17:1 or more, 18:1 or more, 19:1 or more, 20:1 or more, 21:1 or more, 22:1 or more, 23:1 or more, or 24:1 or more, and the weight ratio of the first composition to the second composition may also be 25:1 or less, 24:1 or less, 23:1 or less, 22:1 or less, 21:1 or less, 20:1 or less, 19:1 or less, 18:1 or less, 17:1 or less, or 16:1 or less.

In one embodiment, the mixing may include, for example, not only directly rubbing by using a hand but also shaking a container containing the first composition and the second composition together.

A cosmetic kit according to one embodiment of the present invention may change properties to a cream form only by combining the first composition and the second composition into one container immediately before use and shaking several times, for example, 1 to 5 times, 1 to 4 times, 1 to 3 times, or 1 to 2 times.

In addition, the cosmetic kit according to one embodiment of the present invention may be used as a cosmetic composition of a cream formulation by mixing the first composition and the second composition, and as necessary, the first composition of a nanoemulsion form of a liquid phase may be used alone as a cosmetic composition of a toner formulation.

In one embodiment, the first composition may include wax and oil.

In one embodiment, the wax may be one or more selected from the group consisting of cetyl esters, polyglyceryl-3 methylglucose distearate, cetyl palmitate, cetyl alcohol, olive wax, beeswax, carnauba wax, candelilla wax, microcrystalline wax, and jojoba esters.

In one embodiment, the wax may be included in an amount of 5 to 20 wt % based on a total weight of the first composition. Specifically, the wax may be included in an amount of 5 wt % or more, 6 wt % or more, 7 wt % or more, 8 wt % or more, 9 wt % or more, 10 wt % or more, 11 wt % or more, 12 wt % or more, 13 wt % or more, 14 wt % or more, 15 wt % or more, 16 wt % or more, 17 wt % or more, or 18 wt % or more based on the total weight of the first composition, and the wax may also be included in an amount of 20 wt % or less, 19 wt % or less, 18 wt % or less, 17 wt % or less, 16 wt % or less, 15 wt % or less, 14 wt % or less, 13 wt % or less, 12 wt % or less, 11 wt % or less, 10 wt % or less, 9 wt % or less, 8 wt % or less, 7 wt % or less, or 6 wt % or less based on the total weight of the first composition.

In one embodiment, the oil may be an ester-based oil.

In one embodiment, the ester-based oil may be one or more selected from the group consisting of dicaprylyl carbonate, isopropyl myristate, isopropyl palmitate, octyl-dodecyl myristate, cetyl ethylhexanoate, C14-18 alkyl ethylhexanoate, coco-caprylate/caprate, decyl cocoate, butylene glycol dicaprylate/dicaprate, diisostearyl malate, and di-C12-13 alkyl malate.

In one embodiment, the oil may be included in an amount of 3 to 10 wt % based on a total weight of the first composition. Specifically, the oil may be included in an amount of 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, or 9.5 wt % or more based on a total weight of the first composition, and the oil may also be included in an amount of 10 wt % or less, 9.5 wt % or less, 9 wt % or less, 8.5 wt % or less, 8 wt % or less, 7.5 wt % or less, 7 wt % or less, 6.5 wt % or less, 6 wt % or less, 5.5 wt % or less, 5 wt % or less, 4.5 wt % or less, 4 wt % or less, or 3.5 wt % or less based on the total weight of the first composition.

In one embodiment, the first composition may further include a polyol. In one embodiment, the polyol is not particularly limited as long as it is commonly used in a cosmetic composition, and for example, may be selected from the group consisting of propylene glycol, dipropylene glycol, butylene glycol, glycerin, ethylhexylglycerin, 1,3-propanediol, isoprene glycol, pentylene glycol, 1,2-hexanediol, erythritol, xylitol, sorbitol, and mixtures thereof.

In one embodiment, the first composition may further include a moisturizer, a UV absorber, a UV screening agent, a preserving agent, an osmosis agent, a buffer, a stabilizer, a surfactant, a preservative, or the like, and also, the first composition may further include a conventional additive such as an antioxidant, a stabilizing agent, a solubilizer, a vitamin, a pigment, and a fragrance.

In one embodiment, the second composition may include one or more of a salt or a pH reducing agent, and water.

In one embodiment, the salt may be one or more selected from sodium chloride (NaCl) and magnesium sulfate (MgSO₄).

In one embodiment, the salt may be included in an amount of 10 to 30 wt % based on a total weight of the second composition. Specifically, the salt may be included in an amount of 10 wt % or more, 12 wt % or more, 14 wt % or more, 16 wt % or more, 18 wt % or more, 20 wt % or more, 22 wt % or more, 24 wt % or more, 26 wt % or more, or 28 wt % or more based on the total weight of the second composition, and the salt may also be included in an amount of 30 wt % or less, 28 wt % or less, 26 wt % or less, 24 wt % or less, 22 wt % or less, 20 wt % or less, 18 wt % or less, 16 wt % or less, 14 wt % or less, or 12 wt % or less based on the total weight of the second composition.

In one embodiment, the pH reducing agent may be one or more selected from a poly-hydroxy-acid (PHA) and an alpha-hydroxy-acid (AHA).

In one embodiment, the poly-hydroxy-acid may be gluconolactone.

In one embodiment, the alpha-hydroxy-acid (AHA) may be one or more selected from glycolic acid, lactic acid, and mandelic acid.

In one embodiment, the pH reducing agent may be included in an amount of 10 to 30 wt % based on the total weight of the second composition. Specifically, the pH reducing agent may be included in an amount of 10 wt % or more, 12 wt % or more, 14 wt % or more, 16 wt % or more, 18 wt % or more, 20 wt % or more, 22 wt % or more, 24 wt % or more, 26 wt % or more, or 28 wt % or more based on the total weight of the second composition, and the pH reducing agent may also be included in an amount of 30 wt % or less, 28 wt % or less, 26 wt % or less, 24 wt % or less, 22 wt % or less, 20 wt % or less, 18 wt % or less, 16 wt % or less, 14 wt % or less, or 12 wt % or less based on the total weight of the second composition.

In one embodiment, the first composition may be included in an amount of 90 to 97 wt % based on a total weight of the first composition and the second composition. Specifically, the first composition may be included in an amount of 90 wt % or more, 91 wt % or more, 92 wt % or more, 93 wt % or more, 94 wt % or more, 95 wt % or more, or 96 wt % or more based on the total weight of the first composition and the second composition, and the first composition may also be included in an amount of 97 wt % or less, 96 wt % or less, 95 wt % or less, 94 wt % or less, 93 wt % or less, 92 wt % or less, or 91 wt % or less based on the total weight of the first composition and the second composition.

In one embodiment, the second composition may be included in an amount of 3 to 10 wt % based on the total weight of the first composition and the second composition. Specifically, the second composition may be included in an amount of 3 wt % or more, 4 wt % or more, 5 wt % or more, 6 wt % or more, 7 wt % or more, 8 wt % or more, or 9 wt % or more based on the total weight of the first composition and the second composition, and the second composition may also be included in an amount of 10 wt % or less, 9 wt % or less, 8 wt % or less, 7 wt % or less, 6 wt % or less, 5 wt % or less, or 4 wt % or less based on the total weight of the first composition and the second composition.

The present invention, in an example, may provide the following embodiments.

First embodiment may provide a cosmetic kit comprising: a first composition of a nanoemulsion form of a liquid phase including wax and oil; and a second composition of a liquid phase including one or more of a salt or a pH reducing agent, and water, in which the first composition and the second composition are separated from each other.

Second embodiment, in the first embodiment, may provide the cosmetic kit in which when the first composition and the second composition are mixed, a mixed composition may change properties from a liquid toner form to a cream form.

Third embodiment, in one or more of the first embodiment or the second embodiment, may provide the cosmetic kit in which a hardness of the mixed composition of a cream form is 10 to 70 gf/cm².

Fourth embodiment, in one or more of the first embodiment to the third embodiment, may provide the cosmetic kit in which the wax is included in an amount of 5 to 20 wt % based on a total weight of the first composition.

Fifth embodiment, in one or more of the first embodiment to the fourth embodiment, may provide the cosmetic kit in which the oil is an ester-based oil.

Sixth embodiment, in one or more of the first embodiment to the fifth embodiment, may provide the cosmetic kit in which the oil is included in an amount of 3 to 10 wt % based on a total weight of the first composition.

Seventh embodiment, in one or more of the first embodiment to the sixth embodiment, may provide the cosmetic kit in which the first composition and the second composition are mixed in a weight ratio of 15-25:1.

Eighth embodiment, in one or more of the first embodiment to the seventh embodiment, may provide the cosmetic kit in which the salt is one or more selected from sodium chloride (NaCl) and magnesium sulfate (MgSO₄).

Ninth embodiment, in one or more of the first embodiment to the eighth embodiment, may provide the cosmetic kit in which the pH reducing agent is one or more selected from a poly-hydroxy-acid (PHA) and an alpha-hydroxy-acid (AHA).

Tenth embodiment, in one or more of the first embodiment to the ninth embodiment, may provide the cosmetic kit in which the poly-hydroxy-acid (PHA) is gluconolactone.

Eleventh embodiment, in one or more of the first embodiment to the tenth embodiment, may provide the cosmetic kit in which the alpha-hydroxy-acid (AHA) is one or more selected from glycolic acid, lactic acid, and mandelic acid.

Twelfth embodiment, in one or more of the first embodiment to the eleventh embodiment, may provide the cosmetic kit in which the first composition is included in an amount of 90 to 97 wt % based on a total weight of the first composition and the second composition.

Thirteenth embodiment, in one or more of the first embodiment to the twelfth embodiment, may provide the cosmetic kit in which the second composition is included in an amount of 3 to 10 wt % based on a total weight of the first composition and the second composition.

Fourteenth embodiment may provide a method for skin care using the cosmetic kit according to one or more of the first embodiment to the thirteenth embodiment, the method comprising: after applying the first composition of the cosmetic kit of one or more of the first embodiment to the thirteenth embodiment to skin, applying a second composition to the skin and mixing while rubbing; or, after mixing the first composition and the second composition of the cosmetic kit of one or more of the first embodiment to the thirteenth embodiment, applying to skin.

Fifteenth embodiment, in the fourteenth embodiment, may provide the method in which when the first composition and the second composition are mixed, a mixed composition may change properties from a liquid toner form to a cream form.

Sixteenth embodiment, in one or more of the fourteenth embodiment and the fifteenth embodiment, may provide the method in which the first composition and the second composition are mixed in a weight ratio of 15-25:1.

Seventeenth embodiment, in one or more of the fourteenth embodiment to the sixteenth embodiment, may provide the method in which a hardness of the mixed composition of a cream form is 10 to 70 gf/cm².

Eighteenth embodiment, in one or more of the fourteenth embodiment to the sixteenth embodiment, may provide the method in which the wax is included in an amount of 5 to 20 wt % based on a total weight of the first composition, or the oil is included in an amount of 3 to 10 wt % based on the total weight of the first composition.

Nineteenth embodiment, in one or more of the fourteenth embodiment to the sixteenth embodiment, may provide the method in which the oil is an ester-based oil, or the salt is one or more selected from sodium chloride (NaCl) and magnesium sulfate (MgSO₄), or the pH reducing agent is one or more selected from a poly-hydroxy-acid (PHA) and an alpha-hydroxy-acid (AHA).

Twentieth embodiment, may provide the method in which the poly-hydroxy-acid (PHA) is gluconolactone, or the alpha-hydroxy-acid (AHA) is one or more selected from glycolic acid, lactic acid, and mandelic acid.

Hereinafter, contents of the present invention will be described more specifically through examples and test examples. However, these examples and test examples are presented for the purpose of understanding the content of the present invention, and the scope of the present invention is not limited to these examples and test examples, and modifications, substitutions, insertions, and the like known in the art can be made, which are also included in the scope of the present invention.

### [Manufacturing Example] Manufacturing of First and Second Compositions

### 1. Manufacturing of First Composition

The first composition was manufactured with a composition and a content (wt % based on a total weight of the first composition and the second composition) of Table 1 below. Specifically, oil/wax components of cetyl esters, polyglyceryl-3 methylglucose distearate, and dicaprylyl carbonate were heated to 60 to 70° C., and the remaining betaine, sodium stearoyl glutamate, glycerin, butylene glycol, ethylhexylglycerin, 1,2-hexanediol, and water were heated to 70° C., and then after emulsifying primarily at 5000 rpm through homomixing with the oil/wax components, each of the first compositions was manufactured by performing high-pressure emulsification three times at 1000 bar using a high-pressure emulsifier (Microfluidizer, Songwon System).

TABLE 1

| | Manufacturing | Manufacturing | Manufacturing | Manufacturing | Manufacturing |
|---|---|---|---|---|---|
| Components | Example 1 | Example 2 | Example 3 | Example 4 | Example 5 |
| Cetyl esters | 9 | 6 | 15 | 3 | 20 |
| Polyglyceryl-3 | 3 | 2 | 5 | 1 | 8 |
| methylglucose | | | | | |
| distearate | | | | | |
| Dicaprylyl | 6 | 4 | 9 | 2 | 12 |
| carbonate | | | | | |
| Betaine | 2 | 2 | 2 | 2 | 2 |
| Sodium stearoyl | 0.2 | 0.2 | 0.2 | 0.2 | 0.2 |
| glutamate | | | | | |
| Glycerin | 5 | 5 | 5 | 5 | 5 |
| Butylene glycol | 12 | 12 | 12 | 12 | 12 |
| Ethylhexylglycerin | 0.05 | 0.05 | 0.05 | 0.05 | 0.05 |
| 1,2-hexanediol | 2 | 2 | 2 | 2 | 2 |
| Water | balance | balance | balance | balance | balance |

### 2. Manufacturing of Second Composition

The second composition was manufactured by mixing with a composition and a content (wt % based on a total weight of the first composition and the second composition) of Table 2 below.

TABLE 2

| | Manufacturing | Manufacturing | Manufacturing |
|---|---|---|---|
| Components | Example 6 | Example 7 | Example 8 |
| Water | 4 | 4 | 4 |
| NaCl | 1 | | |
| Gluconolactone | | 1 | |
| MgSO₄ | | | 1 |

### EXAMPLES AND COMPARATIVE EXAMPLES

By combining the first composition and the second composition of the manufacturing example in combinations of Table 3 below (mixing weight ratio 19:1), a hardness of the composition at the time of mixing (after rubbing by hand) was evaluated using a Sun Rheometer, and results thereof were shown together in Table 3.

TABLE 3

| | | | | | | Comparative | Comparative |
|---|---|---|---|---|---|---|---|
| | Example 1 | Example 2 | Example 3 | Example 4 | Example 5 | Example 1 | Example 2 |
| First | Manufacturing | Manufacturing | Manufacturing | Manufacturing | Manufacturing | Manufacturing | Manufacturing |
| composition | Example 1 | Example 2 | Example 3 | Example 1 | Example 1 | Example 4 | Example 5 |
| Second | Manufacturing | Manufacturing | Manufacturing | Manufacturing | Manufacturing | Manufacturing | Manufacturing |
| composition | Example 6 | Example 6 | Example 6 | Example 7 | Example 8 | Example 6 | Example 6 |
| Hardness | 25 | 10 | 65 | 26 | 21 | — | — |
| (gf/cm²) | | | | | | | |

As shown in Table 3, in case of Examples 1 to 5 in which the first compositions of Manufacturing Examples 1 to 3 and the second compositions of Manufacturing Examples 6 to 8, according to one embodiment of the present invention, were mixed, it was confirmed that a stable properties change occurred from a light formulation cream (hardness 10 gf/cm²) to a rich formulation cream (hardness 65 gf/cm²). On the other hand, in case of using the first compositions of Manufacturing Examples 4 and 5 which were out of the content ranges of wax and oil according to the present invention, it was confirmed that a stability of the first composition was not good, thereby causing a phase separation (Manufacturing Example 4), or not being maintained in a liquid phase and changing to a cream form (Manufacturing Example 5).

### [Test Example 1] Evaluation of Properties Change

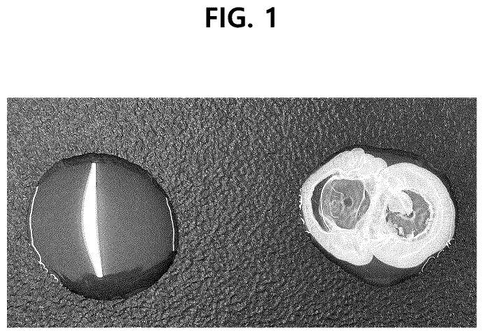

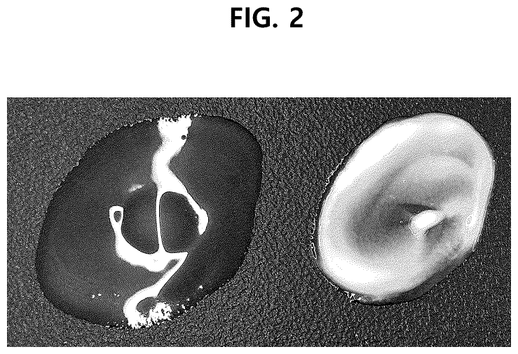

In case of immediately after dropping the second composition of Manufacturing Example 6 into the first composition of Manufacturing Example 1 (weight ratio 19:1) and physically mixing (rubbing by hand), a properties change of the composition was visually confirmed, and results thereof were shown in FIG. 1 and FIG. 2.

As shown in FIG. 1, in case of the first composition alone, it was a toner formulation of a liquid phase (left), but it was confirmed that immediately after dropping the second composition into the first composition, a properties change to a cream formulation occurred (right).

In addition, as shown in FIG. 2, in case of rubbing each composition five times by hand, it was confirmed that in case of the first composition alone (left), almost no properties change occurred, while in case of dropping the second composition into the first composition, only by rubbing five times, a stable cream formulation was formed.

### [Test Example 2] Evaluation of Properties Change when Applied to Skin

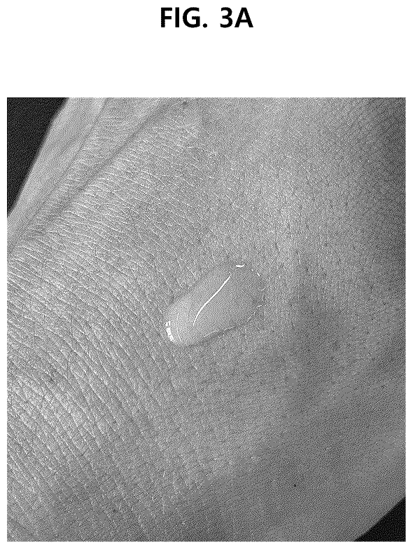

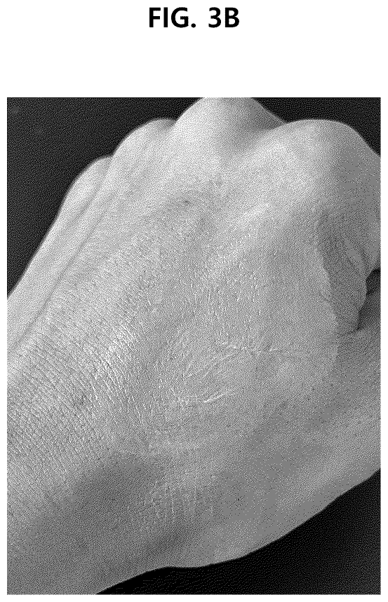

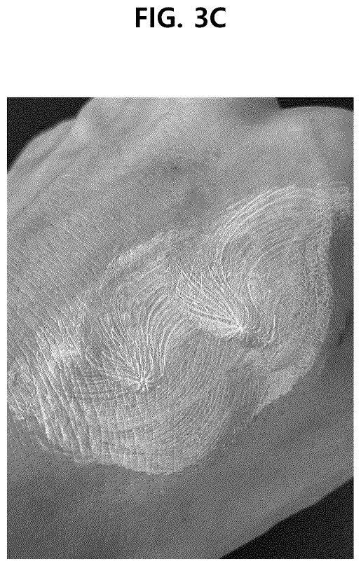

Immediately after dropping two drops of the first composition of Manufacturing Example 1 on a back of a hand (FIG. 3A), after rubbing by hand five times (FIG. 3B), and after rubbing by hand twenty times (FIG. 3C), a properties change was visually confirmed for each, and results thereof were shown in FIGS. 3A to 3C.

As shown in FIGS. 3A to 3C, in case of the first composition alone, only by rubbing five times, almost no properties change occurred, and about twenty times rubbing was required to show a creamifying tendency, and even in this case, it was confirmed that a stable cream formulation was not formed and a properties change occurred to a lotion-like formulation.

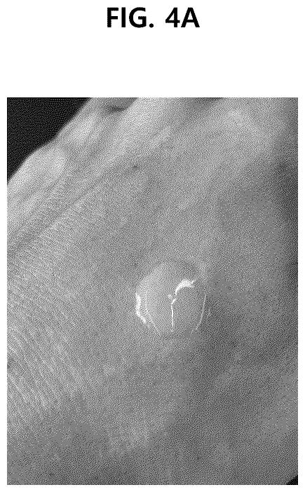

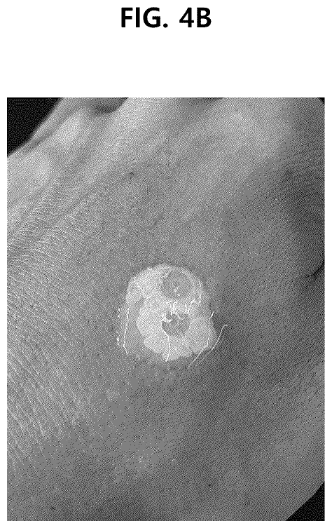

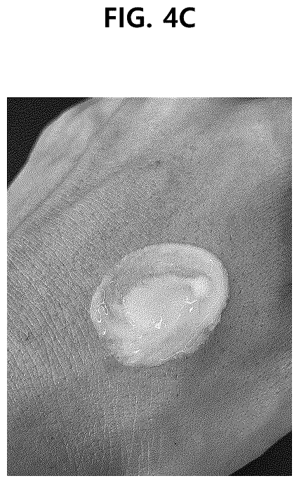

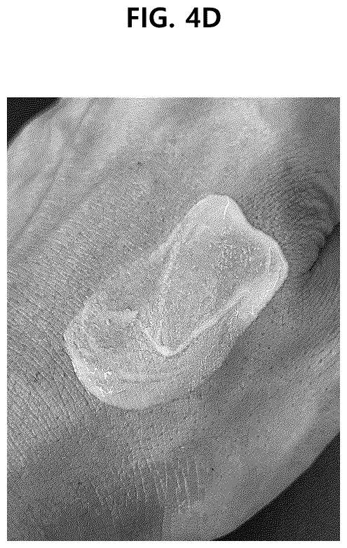

On the other hand, compared to the case of the first composition of Manufacturing Example 1 alone (FIG. 4A), immediately after dropping the second composition of Manufacturing Example 6 into the first composition (weight ratio: 19:1) (FIG. 4B), after rubbing by hand five times (FIG. 4C), and after rubbing by hand ten times (FIG. 4D), a properties change was visually confirmed for each, and results thereof were shown in FIGS. 4A to 4D.

As shown in FIGS. 4A to 4D, it was confirmed that immediately after dropping the second composition into the first composition, a properties change to a cream formulation began to occur (FIG. 4B), and only by rubbing five times and ten times, a soft cream formulation was stably formed (FIGS. 4C and 4D).

We claim:

1. A cosmetic kit, comprising:
a first composition of a nanoemulsion form of a liquid phase including wax and oil; and
a second composition of a liquid phase including one or more of a salt or a pH reducing agent, and water,
wherein the first composition and the second composition are separated from each other.

2. The cosmetic kit of claim 1, wherein when the first composition and the second composition are mixed, a mixed composition changes properties from a liquid toner form to a cream form.

3. The cosmetic kit of claim 2, wherein a hardness of the mixed composition of the cream form is 10 to 70 gf/cm².

4. The cosmetic kit of claim 1, wherein the wax is included in an amount of 5 to 20 wt % based on a total weight of the first composition.

5. The cosmetic kit of claim 1, wherein the oil is an ester-based oil.

6. The cosmetic kit of claim 1, wherein the oil is included in an amount of 3 to 10 wt % based on a total weight of the first composition.

7. The cosmetic kit of claim 1, wherein the first composition and the second composition are mixed in a weight ratio of 15-25:1.

8. The cosmetic kit of claim 1, wherein the salt is one or more selected from sodium chloride (NaCl) and magnesium sulfate (MgSO₄).

9. The cosmetic kit of claim 1, wherein the pH reducing agent is one or more selected from a poly-hydroxy-acid (PHA) and an alpha-hydroxy-acid (AHA).

10. The cosmetic kit of claim 9, wherein the poly-hydroxy-acid (PHA) is gluconolactone.

11. The cosmetic kit of claim 9, wherein the alpha-hydroxy-acid (AHA) is one or more selected from glycolic acid, lactic acid, and mandelic acid.

12. The cosmetic kit of claim 1, wherein the first composition is included in an amount of 90 to 97 wt % based on a total weight of the first composition and the second composition.

13. The cosmetic kit of claim 1, wherein the second composition is included in an amount of 3 to 10 wt % based on a total weight of the first composition and the second composition.

14. A method for skin care using the cosmetic kit of claim 1, the method comprising:
after applying the first composition of the cosmetic kit to skin, applying the second composition to the skin and mixing while rubbing; or
after mixing the first composition and the second composition of the cosmetic kit, applying to skin.

15. The method of claim 14, wherein when the first composition and the second composition are mixed, a mixed composition changes properties from a liquid toner form to a cream form.

16. The method of claim 14, wherein the first composition and the second composition are mixed in a weight ratio of 15-25:1.

17. The method of claim 15, wherein a hardness of the mixed composition of the cream form is 10 to 70 gf/cm².

18. The method of claim 14, wherein the wax is included in an amount of 5 to 20 wt % based on a total weight of the first composition, or the oil is included in an amount of 3 to 10 wt % based on the total weight of the first composition.

19. The method of claim 14, wherein the oil is an ester-based oil, or the salt is one or more selected from sodium chloride (NaCl) and magnesium sulfate (MgSO₄), or the pH reducing agent is one or more selected from a poly-hydroxy-acid (PHA) and an alpha-hydroxy-acid (AHA).

20. The method of claim 19, wherein the poly-hydroxy-acid (PHA) is gluconolactone, or the alpha-hydroxy-acid (AHA) is one or more selected from glycolic acid, lactic acid, and mandelic acid.
